SEQUENCE LISTING (30 sequences)

SEQ ID NO: 1 - DNA, 20 bp, synthetic construct
  source 1..20: mol_type = other DNA; note = alpha-Casein S1 Forward
aagagggaatccatgcccaa

SEQ ID NO: 2 - DNA, 21 bp, synthetic construct
  source 1..21: mol_type = other DNA; note = alpha-Casein S1 Reverse
aggccagttcctgattcactc

SEQ ID NO: 3 - DNA, 19 bp, synthetic construct
  source 1..19: mol_type = other DNA; note = DELTA 1 (DLL1) Forward
agggccagtactgcacaga

SEQ ID NO: 4 - DNA, 19 bp, synthetic construct
  source 1..19: mol_type = other DNA; note = DELTA 1 (DLL1) Reverse
aaaatccgtgctgctcatc

SEQ ID NO: 5 - DNA, 19 bp, synthetic construct
  source 1..19: mol_type = other DNA; note = ERalpha (ESR1) Forward
cgcaagtgctatgaagtgg

SEQ ID NO: 6 - DNA, 19 bp, synthetic construct
  source 1..19: mol_type = other DNA; note = ERalpha (ESR1) Reverse
cgcttgtgcttcaacattc

SEQ ID NO: 7 - DNA, 19 bp, synthetic construct
  source 1..19: mol_type = other DNA; note = STAT5A Forward
gcagccatctcgaggacta

SEQ ID NO: 8 - DNA, 19 bp, synthetic construct
  source 1..19: mol_type = other DNA; note = STAT5A Reverse
gaaccactgccagaaggtg

SEQ ID NO: 9 - DNA, 20 bp, synthetic construct
  source 1..20: mol_type = other DNA; note = alpha-lactalbumin Forward
ctccaggggtgcatgaatgg

SEQ ID NO: 10 - DNA, 21 bp, synthetic construct
  source 1..21: mol_type = other DNA; note = alpha-lactalbumin Reverse
taaaagcgccatcagggacat

SEQ ID NO: 11 - DNA, 20 bp, synthetic construct
  source 1..20: mol_type = other DNA; note = BLG Forward
gggaaaaccacgagtgtgtg

SEQ ID NO: 12 - DNA, 21 bp, synthetic construct
  source 1..21: mol_type = other DNA; note = BLG Reverse
tttccccgtgatagttgaccg

SEQ ID NO: 13 - DNA, 20 bp, synthetic construct
  source 1..20: mol_type = other DNA; note = EIF4E Forward
caggaggttgctaacccaga

SEQ ID NO: 14 - DNA, 19 bp, synthetic construct
  source 1..19: mol_type = other DNA; note = EIF4E Reverse
tgcttgccaagttttgctt

SEQ ID NO: 15 - DNA, 19 bp, synthetic construct
  source 1..19: mol_type = other DNA; note = Ki67 Forward
gtcagcagcttcggtgatt

SEQ ID NO: 16 - DNA, 18 bp, synthetic construct
  source 1..18: mol_type = other DNA; note = Ki67 Reverse
gagacccgcctcctcttt

SEQ ID NO: 17 - DNA, 19 bp, synthetic construct
  source 1..19: mol_type = other DNA; note = Notch1 Forward
aacgagttcgtgtgcgagt

SEQ ID NO: 18 - DNA, 19 bp, synthetic construct
  source 1..19: mol_type = other DNA; note = Notch1 Reverse
gttcttgcagggtgtgctt

SEQ ID NO: 19 - DNA, 19 bp, synthetic construct
  source 1..19: mol_type = other DNA; note = Jagged 1 Forward
tcccactggtttctctgga

SEQ ID NO: 20 - DNA, 19 bp, synthetic construct
  source 1..19: mol_type = other DNA; note = Jagged 1 Reverse
ctggctcggttgtagcact

SEQ ID NO: 21 - DNA, 22 bp, synthetic construct
  source 1..22: mol_type = other DNA; note = LGR4 Forward
gggcttaaaagcacctgtcgaa

SEQ ID NO: 22 - DNA, 21 bp, synthetic construct
  source 1..21: mol_type = other DNA; note = LGR4 Reverse
aggtctcagtgatgtggcagg

SEQ ID NO: 23 - DNA, 20 bp, synthetic construct
  source 1..20: mol_type = other DNA; note = LGR5 Forward
agttgttcagcctccgatct

SEQ ID NO: 24 - DNA, 20 bp, synthetic construct
  source 1..20: mol_type = other DNA; note = LGR5 Reverse
tggaaaatgcattagggtca

SEQ ID NO: 25 - DNA, 20 bp, synthetic construct
  source 1..20: mol_type = other DNA; note = S16 Forward
atctcgtctctgcgccttga

SEQ ID NO: 26 - DNA, 20 bp, synthetic construct
  source 1..20: mol_type = other DNA; note = S16 Reverse
gcattcttctgccggtgtcg

SEQ ID NO: 27 - DNA, 20 bp, synthetic construct
  source 1..20: mol_type = other DNA; note = LGR6 Forward
gtgtcctggagctgtctcac

SEQ ID NO: 28 - DNA, 20 bp, synthetic construct
  source 1..20: mol_type = other DNA; note = LGR6 Reverse
ctcctccagcttctgacacc

SEQ ID NO: 29 - DNA, 22 bp, synthetic construct
  source 1..22: mol_type = other DNA; note = Beta-casein Forward
ttccattgcctggactacttgt

SEQ ID NO: 30 - DNA, 20 bp, synthetic construct
  source 1..20: mol_type = other DNA; note = Beta-casein Reverse
caacagccaaaaggcaggta

(19) United States
(12) Patent Application Publication
(10) Pub. No.: US 2024/0423246 A1
(43) Pub. Date: Dec. 26, 2024

(54) RAPAMYCIN FOR INCREASED MILK PRODUCTION

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventors: Itamar BARASH, Rohovot (IL); Anna KOSENKO, Ramla (IL); Maya ROSS, Rohovot (IL)

(73) Assignee: THE STATE OF ISRAEL, MINISTRY OF AGRICULTURE & RURAL DEVELOPMENT, AGRICULTURAL RESEARCH ORGANIZATION, Rishon Lezion (IL)

(21) Appl. No.: 18/338,382

(22) Filed: Jun. 21, 2023

(51) Int. Cl. A23K 50/10; A23K 20/121; A23K 20/184; A61K 31/436; A61K 31/57

(52) U.S. Cl. CPC A23K 50/10; A23K 20/121; A23K 20/184; A61K 31/436; A61K 31/57

(57) ABSTRACT

The present disclosure provides methods of enhancing milk production in animals comprising administering to the animal a milk-secretion stimulating amount of rapamycin or a rapamycin analogue.

### SEQUENCE LISTING STATEMENT

The instant application contains a Sequence Listing which has been submitted electronically in XML file format and is hereby incorporated by reference in its entirety. Said XML copy, created on Jun. 21, 2023, is named P-627433-US_SL1.xml and is 32,643 bytes in size.

### FIELD OF INTEREST

The present disclosure relates to methods of increasing milk production in animals comprising administering to the animal a milk-secretion stimulating amount of rapamycin or a rapamycin analogue. Further disclosed are methods of increasing milk protein expression in animals.

### BACKGROUND

Mammary epithelial stem cells give rise to the basal and luminal layers that compose the functional mammary epithelium, and renew the mammary gland between consecutive lactation periods.

Notable progress has been achieved in mouse mammary stem cell characterization since breakthrough studies were published, demonstrating reconstitution of a functional mammary gland by transplantation of a single or several stem cells into the de-epithelized mammary stroma (Shackleton M. et al., Nature. 2006; 439:84-8; Stingl J. et al., Nature. 2006; 439:993-7). Together with their bipotent counterparts, these cells maintain the morphogenesis and homeostasis of the adult gland (Rios AC. et al., Nature. 2014; 506:322-7).

Only modest progress has been achieved in bovine mammary stem cell research. It has been demonstrated that the stem cells have the ability to generate a representative bovine mammary morphology in bovine stroma pre-transplanted into the mammary stroma of immunocompromised mice (Kosenko A. et al., Cell Tissue Res. 2022; 39-61).

In an attempt to induce bovine mammary epithelial stem cell number, 3-month-old calves received intramammary xanthosine infusion for 5 consecutive days (Capuco AV. et al., Experimental biology and medicine. 2009; 234:475-82). An elevated number of retained BrdU-labeled epithelial cells and induced telomerase activity were noted. Later, induced expression of stem cell gene markers by xanthosine was also observed in goats (Choudhary RK. et al., Molecular biology reports. 2018; 45:581-90). Nevertheless, xanthosine's self-renewing effect could not be reproduced in stem cells of transplanted bovine parenchyma implants, which were analyzed for BrdU-cell retention and by flow cytometry (Rauner G. et al., 2014; 328:186-96). Moreover, xanthosine administration suppressed bovine mammary cell proliferation by 50%, due to inhibition of inosine-5′-monophosphate dehydrogenase activity, a rate-limiting enzyme in guanine synthesis.

Rapamycin inhibits mammalian target of rapamycin (mTOR) and has immunosuppressant functions and antiproliferative properties in humans and is especially useful in preventing organ transplant rejection.

Methods for increasing the milk production of animals in the dairy industry are required.

### SUMMARY OF THE DISCLOSURE

In one aspect, disclosed herein is a method of increasing the milk production of an animal, the method comprising administering to the animal a milk-secretion stimulating amount of a mTOR inhibitor.

In another aspect, disclosed herein is a method of increasing the level of milk proteins in an animal, the method comprising administering to the animal a milk-protein stimulating amount of a mTOR inhibitor.

In a related aspect, the mTOR inhibitor comprises rapamycin or a rapamycin analogue.

In a related aspect, the the mTOR inhibitor is administered by intramammary infusion.

In a related aspect, the mTOR inhibitor is administered at a dose of between 1 mg to 30 mg.

In a related aspect, the animal is further administered estrogen and progesterone.

In a related aspect, the milk protein is selected from the group consisting of α-S1-casein (alpha-S1-casein), α-S2-casein (alpha-S2-casein), β-casein (beta-casein), K-casein (kappa-casein), β-lactoglobulin (BLG), and α-lactalbumin (alpha-lactalbumin).

In a related aspect, the animal is a mammal. In a related aspect, the animal is a non-lactating animal. In a related aspect, the animal is a dairy heifer or a non-pregnant cow. In a related aspect, the animal is a dairy livestock animal. In a related aspect, the livestock animal is selected from the group consisting of a heifer, a cow, a goat, a sheep and a water buffalo. In a related aspect, the animal is a calf. In a related aspect, the animal is a 3-month-old calf.

In one aspect, disclosed herein is a method for inducing epithelial cell proliferation in the mammary gland of an animal comprising administering to the animal a cell proliferation stimulating amount of a mTOR inhibitor.

In a related aspect, the epithelial cells comprise luminal epithelial cells. In a related aspect, the epithelial cells comprise myoepithelial (basal) cells.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1: schematic designation of the bovine mammary glands for intramammary vehicle and rapamycin administration.

FIGS. 2A-2L: (FIG. 2A) Mammary gland morphology; P=parenchyma; S=stroma; Bar=2 cm. (FIG. 2B) Evans blue penetration of parenchyma, 2h after administration; Bar=1.25 cm. (FIG. 2C) Mammary whole mount showing penetration of Evans blue into Carmine red-stained mammary epithelial structures; Bar=1 mm. (FIG. 2D) Immunoblot analysis showing a decrease in S6 phosphorylation (pS6) 24 and 48h after rapamycin administration. FIGS. 2E-2G: Immunohistochemical analysis of S6 phosphorylation, 24 h after treatment, in regions close to the nipple: (FIG. 2E) Vehicle administration; (FIG. 2F) rapamycin administration (4.16 mg); (FIG. 2G) rapamycin administration (8.32 mg). FIGS. 2H-2L: Immunohistochemical analysis of S6 phosphorylation, 48 h after treatment, in regions close to and far from the nipple: (FIG. 2H) Vehicle administration; (FIG. 2I) Rapamycin administration (4.16 mg), close to nipple; (FIG. 2J) rapamycin administration (8.32 mg), close to nipple; (FIG. 2K) Rapamycin administration (4.16 mg), far from nipple; (FIG. 2L) Rapamycin administration (8.32 mg), far from nipple. Bar=50 μm.

FIGS. 3A-3F: (FIG. 3A) Immunoblot analysis of phosphorylated S6 (pS6) and β-casein. One of the nipples in calf #2 was inaccessible. (FIG. 3B) Rapamycin administration causes a decrease in pS6 expression relative to β-actin expression. (FIG. 3C) Rapamycin administration does not affect β-casein expression relative to β-actin expression. Bar graphs represent mean±SEM of 3-4 replications. (FIG. 3D) H&E-stained mammary parenchymal sections demonstrating no effect of rapamycin on mammary morphology. Insets: higher magnifications. (FIG. 3E) Representative immunohistochemical analysis demonstrating lower S6 phosphorylation in rapamycin-treated vs. vehicle-treated glands. Bar=50 μm. (FIG. 3F) Representative immunohistochemical analysis demonstrating no effect of rapamycin administration on β-casein expression in the mammary epithelial cells. Bar=50 μm.

FIG. 4A-4B: (FIG. 4A) Dispersed cells isolated from individual vehicle- and rapamycin-treated mammary glands were seeded in 96-well plates and cultured in mammary medium. At the indicated time points (arrows), cells were trypsinized, counted and reseeded. Inset: cell number at the end of the culture period. Bars represent mean±SEM. (FIG. 4B) Relative gene expression analysis of luminal progenitor marker (Stat5) and stem cell markers (Notch1, Jagged1, Delta1, LGR4, LGR5). N=4 for vehicle-treated and 3 for rapamycin-treated glands. Six wells of cells from each gland were analyzed.

FIGS. 5A-5D: (FIG. 5A) Representative H&E staining of vehicle- and rapamycin-treated glands of 3-month-old vehicle-treated and rapamycin-treated bovine mammary gland after 4-day treatment with estrogen and progesterone. Bar=50 μm. (FIG. 5B) Immunofluorescence analysis demonstrating multilayered luminal cells stained with CK18 (green) and a single layer of basal myoepithelial cells stained with aSMA (red). Bar=20 μm. (FIG. 5C) Density of basal cells in the ductal perimeter is higher in large vs. small ducts of the vehicle-treated gland. (FIG. 5D) Number of PCNA+ cells is higher in small vs. large ducts of the vehicle-treated gland, mainly due to difference in luminal cell proliferation. Six independent microscopic fields (X10 magnification) from each of the six glands treated with vehicle were analyzed. Overall, 127 ducts and 10,619 cells were counted. *P<0.05, ** P<0.01, *** P<0.001.

FIGS. 6A-6C: (FIG. 6A) lower and (FIG. 6B) higher magnifications of PCNA-stained parenchymal cells and counterstained with hematoxylin. S, small ducts; L, large ducts. Bar=50 μm. (FIG. 6C) PCNA+ cells were analyzed in total (left plot), basal (middle plot) and luminal cell (right plot) populations of the small and large ducts. Six independent microscopic fields (X10 magnification) from each of the six glands treated with vehicle and six glands treated with rapamycin were analyzed. Overall, 127 ducts and 10,619 cells, and 122 ducts and 9999 cells were analyzed in the vehicle-treated and rapamycin-treated glands, respectively. ** P<0.01, *** P<0.001.

FIGS. 7A-7I: (FIG. 7A) Representative immunofluorescence analyses of milk protein expression by luminal cells in the mammary glands of 3-month-old calves with green staining of β-casein (right) and β-lactoglobulin (BLG) (left) and basal myoepithelial cells stained with aSMA (red). Bar=20 μm. Bar graphs representing effect of rapamycin on relative expression of milk protein genes: (FIG. 7B) aS1-casein, (FIG. 7C) BLG, (FIG. 7D) β-casein, and (FIG. 7E) α-lactalbumin. Bars represent mean #range of two glands in each analysis. (FIG. 7F) Bar graph representing immunoblot analysis of β-casein expression relative to β-actin expression in the mammary glands of vehicle- and rapamycin-treated glands. Bars represent mean±range of two analyzed glands for each calf except for “all”, in which bars represent mean±SEM of N=6 (each) for vehicle- and rapamycin-treated glands. (FIG. 7G) Immunohistochemical analysis supporting the gene-expression analysis and demonstrating higher β-casein expression in epithelial cells of the rapamycin-treated vs. vehicle-treated glands in calf #2. Bar=50 μm. (FIG. 7H) Negative correlation between endogenous levels of milk protein gene expression in the vehicle-treated glands of the individual calves and the respective positive rapamycin effect (fold change). (FIG. 7I) Immunoblot analysis of β-casein and β-actin expression in mammary glands treated with vehicle or rapamycin for 3 weeks followed by estrogen and progesterone administration.

FIGS. 8A-8B: Scheme summarizing the latent effects of intramammary rapamycin administration on cell proliferation and milk protein gene expression. (FIG. 8A) Representative small and large ducts in the developing mammary architecture are boxed. (FIG. 8B) Basal and luminal cell proliferation in small and large ducts is presented in the outer three cell circles, where the relatively lower occupancy of basal cells in the small vs. large ducts can be visualized. Also demonstrated is rapamycin-induced cell proliferation in basal and luminal cells of the small ducts and only in luminal cells of the large ducts. The scheme is a precise representation of the relevant analyses. Milk protein gene expression in the mammary glands of the individual calves is presented in pie chart format, inserted inside the circles showing the proliferation analysis of the large ducts. Presented are Log 2 (N+1) values of milk protein gene expression in the individual calves, calculated to accommodate and visualize the highly different values, some of which were lower than 1. The combined expression values of the four examined milk proteins in the mammary gland of rapamycin-treated calf #1 are highest and represent the maximum potential milk protein gene expression. Respectively, the pie chart demonstrates that rapamycin's inductive effect on milk protein gene expression is limited by the endogenous potential of the individual animal. Thus, almost no improvement in milk protein gene expression is detected in calf #1 with the highest level of endogenously expressed milk proteins. A stronger effect of rapamycin on milk protein gene expression is demonstrated calf #3, and even more so in calf #2 which expressed the lowest level of milk proteins (two orders of magnitude lower than calf #1) and maintained nonutilized expression potential.

### DETAILED DESCRIPTION

The present subject matter may be understood more readily by reference to the following detailed description which forms a part of this disclosure. It is to be understood that this disclosure is not limited to the specific products, methods, conditions or parameters described and/or shown herein, and that the terminology used herein is for the purpose of describing particular embodiments by way of example only and is not intended to be limiting of the claimed disclosure.

It was found that intramammary administration of pharmaceuticals dissolved in a lipophilic vehicle is an effective and efficient method to target mammary epithelial/myoepithelial cells in 3-month-old bovine mammary glands with a potential effect in the adult cow. A decrease of ˜50% in mTOR activity by skip-a-day, 3-week intramammary rapamycin administration is sufficient to induce mammary epithelial stem cell self-renewal with no impairment of mammary morphology or milk protein production. The consequent improvements are induced cell proliferation and milk protein expression and synthesis in glands with relatively low milk protein gene expression. It reflects a genuine latent consequence of the rapamycin administration, followed by a higher number of self-renewed stem cells in the gland.

In some embodiments, disclosed herein is a method of increasing the milk production of an animal, the method comprising administering to the animal a milk-secretion stimulating amount of a mechanistic target of rapamycin (mTOR) inhibitor.

In some embodiments, disclosed herein is a method of increasing the level of milk proteins in an animal, the method comprising administering to the animal a milk-protein stimulating amount of a mTOR inhibitor.

In some embodiments, disclosed herein is a method for inducing epithelial cell proliferation in the mammary gland of an animal comprising administering to the animal a cell proliferation stimulating amount of a mTOR inhibitor.

An artisan would appreciate that the term “mTOR inhibitor” may encompass any substance that inhibits the mechanistic target of rapamycin (mTOR). In some embodiments, the mTOR inhibitor comprises a synthetic inhibitor. In some embodiments, the mTOR inhibitor comprises an inhibitor of mTORC1. In some embodiments, the mTOR inhibitor comprises an inhibitor of mTORC2. In some embodiments, the mTOR inhibitor comprises an inhibitor of both mTORC1 and mTORC2.

In some embodiments, the mTOR inhibitor comprises RMC-6272 (RM-006).

In some embodiments, the mTOR inhibitor comprises rapamycin or a rapamycin analogue. In some embodiments, the mTOR inhibitor comprises rapamycin. In some embodiments, the mTOR inhibitor comprises rapamycin analogue.

In some embodiments, the rapamycin or rapamycin analogue administered to the animal is one which is biologically active in the animal. In some embodiments, the rapamycin or rapamycin analogue may act, at least partially, by increasing the number of differentiating mammary stem cells. This action may be direct, indirect (e.g. through intermediate factors), or both direct and indirect.

The present disclosure provides a method for increasing the milk production of an animal, i.e. effecting mammary secretion of milk from an animal. As used herein “animal” is defined to include all mammals. In some embodiments, the animal is a mammal. In some embodiments, the animal is female. In some embodiments, the method is useful for increasing lactation in animals which have never been pregnant or in animals which are no longer capable of becoming pregnant. In some embodiments, the animal is a dairy heifer or a non-pregnant cow. In some embodiments, the animal is a non-lactating animal. In some embodiments, the animal is a non-pregnant cow. In some embodiments, the animal is a heifer. In some embodiments, the animal is cow. In some embodiments, the animal is mature cow. In some embodiments, the animal is dairy cow. In some embodiments, the animal is lactating cow. In some embodiments, the animal is a non-lactating cow. In some embodiments, the animal is a cow in late lactation (dry period). In some embodiments, the animal is a goat. In some embodiments, the animal is a sheep. In some embodiments, the animal is a water buffalo. In some embodiments, the animal is a dairy livestock animal. In some embodiments, the livestock animal is selected from the group consisting of a heifer, a cow, a goat, a sheep and a water buffalo. In some embodiments, the animal is a calf. In some embodiments, the animal is a 3-month-old calf.

As used herein, the terms “increasing milk production”, “improving milk production”, “enhancing milk production” and “inducing milk production”, all having the same meaning, may encompass improving milk secretion of an animal subsequent to administering a milk-secretion stimulating amount of a mTOR inhibitor, such as rapamycin or a rapamycin analogue. In some embodiments, improved milk secretion is relative compared to a reference value range. For example, in some embodiments, increased milk production is in comparison to the milk production of animals which did not receive a milk-secretion stimulating amount of a mTOR inhibitor, such as rapamycin or a rapamycin analogue. In some embodiments, the increased milk production is at least about 101%, is at least about 105%, is at least about 110%, is at least about 120%, is at least about 130%, is at least about 140%, is at least about 150%, at least about 160%, at least about 170%, at least about 180%, at least about 190%, at least about 200%, at least about 300%, at least about 500%, at least about 700%, at least about 900%, or at least about 1000% greater than the milk production of animals which did not receive a milk-secretion stimulating amount of a mTOR inhibitor, such as rapamycin or a rapamycin analogue. In some embodiments, the term “milk production” refers to the measurable amount of milk, which is extracted from an animal, over a period of time, for example, the amount of milk extracted from an animal over one day (usually over multiple milking sessions), or the average amount of milk extracted from an animal over a period of time, i.e. a week or a month.

In some embodiments, increasing milk production comprises inducing mammary epithelial stem cell self-renewal. In some embodiments, increasing milk production comprises increasing the number of stem cells in the gland. In some embodiments, increasing milk production comprises decreasing mTOR activity. In some embodiments, increasing milk production comprises inhibiting mTOR activity. In some embodiments, increasing milk production comprises inducing cell proliferation. In some embodiments, increasing milk production comprises increased milk protein expression. In some embodiments, increasing milk production comprises increased milk protein synthesis.

As used herein, the term “milk” is the normal mammary secretion of lactating female mammals. As used herein the terms “milk protein” or “milk-protein” may encompass proteins found in milk. Examples of milk proteins include, but are not limited to, β-casein, K-casein, α-S1-casein, α-S2-casein, α-lactalbumin, β-lactoglobulin (BLG), lactoferrin, transferrin, and serum albumin. Additional milk proteins are known in the art.

In some embodiments, milk proteins are selected from the group consisting of serum albumin, alpha-S1-casein, alpha-S2-casein, beta-casein, kappa-casein, beta-lactoglobulin (BLG), and alpha-lactalbumin. In some embodiments, the milk protein is selected from the group consisting of α-S1-casein (alpha-S1-casein), α-S2-casein (alpha-S2-casein), β-casein (beta-casein), K-casein (kappa-casein), β-lactoglobulin (beta-lactoglobulin), and α-lactalbumin (alpha-lactalbumin). In some embodiments, the milk protein comprises β-casein. In some embodiments, the milk protein comprises κ-casein. In some embodiments, the milk protein comprises α-S1-casein. In some embodiments, the milk protein comprises α-S2-casein. In some embodiments, the milk protein comprises α-lactalbumin. In some embodiments, the milk protein comprises β-lactoglobulin (BLG). In some embodiments, the milk protein comprises lactoferrin. In some embodiments, the milk protein comprises transferrin. In some embodiments, the milk protein comprises serum albumin.

In some embodiments, the term “increasing the level of milk proteins”, may encompass increasing the amount of milk proteins in an animal subsequent to administering a milk-protein stimulating amount of a mTOR inhibitor, such as rapamycin or a rapamycin analogue. In some embodiments, increasing the level of milk proteins comprises increasing the levels of milk protein in the glands of the animal. In some embodiments, increasing the level of milk proteins comprises increasing the expression of milk proteins. In some embodiments, increasing the level of milk proteins comprises increasing the expression of milk protein in the glands. One of ordinary skill in the art would appreciate that the term “gene” may encompass a nucleic acid (e.g., DNA or RNA) sequence that comprises coding sequences necessary for the production of RNA, a polypeptide or protein. The term “expression”, as used herein, refers to the production of a functional end-product e.g., an mRNA or a protein.

In some embodiments, increasing the level of milk proteins comprises increasing the total protein content in the milk produced from the animal. It will be appreciated that the “total protein content” is the measurable amount milk proteins the milk produced by an animal. A skilled artisan would be familiar with methods for measuring the protein content in the animal's milk. A skilled artisan would also be familiar with the relative protein content of each milk protein, for example, caseins represent about 80% of total bovine milk proteins, and within the caseins each of the five different types of caseins, namely alpha-S1-casein, alpha-S2-casein, beta-casein, kappa-casein, and gamma-casein, would have their own average protein content in cow milk, for example, 38, 10, 35, and 12%, respectively.

A skilled artisan would appreciate that the term “relative protein content” of a protein may encompass a proportion (or percentage) of that specific protein within the total protein content. In some embodiments, the protein content comprises the protein content found in the animal's milk, such as cow's milk. In some embodiments, increasing the level of milk proteins comprises increasing the total protein content. In some embodiments, increasing the level of milk proteins comprises increasing the total protein content in an animal's milk.

In some embodiments, increasing the level of milk proteins comprises increasing the level of milk proteins in milk produced by the animal. In some embodiments, an increased milk protein level is compared to a reference value range. For example, in some embodiments, the increased milk protein level is compared to the level of milk proteins in animals which did not receive a milk-protein stimulating amount of a mTOR inhibitor, such as rapamycin or a rapamycin analogue. In some embodiments, the level of milk proteins in an animal is measured in the milk produced by that animal. In some embodiments, the level of milk proteins in an animal is measured as the total protein content in the milk produced by that animal.

In some embodiments, the increased milk protein level is at least about 101%, is at least about 105%, is at least about 110%, is at least about 120%, is at least about 130%, is at least about 140%, is at least about 150%, at least about 160%, at least about 170%, at least about 180%, at least about 190%, at least about 200%, at least about 300%, at least about 500%, at least about 700%, at least about 900%, or at least about 1000% greater than the milk protein level of animals which did not receive a milk-protein stimulating amount of a mTOR inhibitor, including rapamycin or a rapamycin analogue.

In some embodiments, the relative protein content of each of the milk proteins is 101%, or up to 150% of the relative protein content of that milk protein in the milk from an animal which did not receive a milk-protein stimulating amount of a mTOR inhibitor, such as rapamycin or a rapamycin analogue.

As used herein the term “inducing epithelial cell proliferation” may encompass increasing the proliferation of epithelial cells of an animal subsequent to administering a cell proliferation stimulating amount of a mTOR inhibitor, including rapamycin or a rapamycin analogue. In some embodiments, inducing epithelial cell proliferation is relative compared to a reference value range. For example, in some embodiments, inducing epithelial cell proliferation is in comparison to the epithelial cell proliferation of animals which did not receive a cell proliferation stimulating amount of a mTOR inhibitor, such as rapamycin or a rapamycin analogue. In some embodiments, inducing epithelial cell proliferation comprises increasing the number of proliferating epithelial cells. In some embodiments, inducing epithelial cell proliferation comprises increasing the number of proliferating epithelial cells in the mammary gland of an animal.

In some embodiments, the number of proliferating epithelial cells is at least about 101%, is at least about 105%, is at least about 110%, is at least about 115%, is at least about 120%, is at least about 130%, is at least about 140%, or is at least about 150%, greater than the number of proliferating epithelial cells of animals which did not receive a cell proliferation stimulating amount of a mTOR inhibitor, such as rapamycin or a rapamycin analogue. In some embodiments, the number of proliferating epithelial cells refers to the measurable number of proliferating cells in an organ. A skilled artisan would be familiar with the methods for measuring such cells, such as those described herein in the Examples.

In some embodiments, inducing epithelial cell proliferation comprises inducing mammary epithelial stem cell self-renewal. In some embodiments, inducing epithelial cell proliferation comprises increasing the number of stem cells in the gland. In some embodiments, inducing epithelial cell proliferation comprises increasing the number of dividing epithelial cells.

An artisan would appreciate that a “cell proliferation stimulating amount” may encompass, in some embodiments, a dosage sufficient to stimulate cell proliferation in the animal to which it is administered.

An artisan would appreciate that the term “proliferating cell” may encompass cells which multiply or reproduce, as a result of cell growth and cell division.

An “epithelial” cell is a cell located in a cellular, avascular layer covering the free surface (cutaneous, mucous or serous) of an organ or lining a tube or cavity of an animal body. In some embodiments, the epithelial cell comprises an epithelial cell in the mammary gland. In some embodiments, the epithelial cell comprises a mammary epithelial cell. In some embodiments, the epithelial cell comprises an epithelial stem cell.

In some embodiments, epithelial cells comprise myoepithelial (basal) cells. In some embodiments, epithelial cells comprise luminal epithelial cells. In some embodiments, epithelial cells comprise both luminal epithelial and myoepithelial (basal) cells.

The term “Rapamycin” may encompass the macrolide compound having the chemical formula C₅₁H₇₉NO₁₃. Rapamycin is also known as “Sirolimus” or “Rapamune” Rapamycin is commercially available.

An artisan would appreciate that the term “rapamycin analogue” (also known as rapalogs) may encompass any natural or synthetic analog of rapamycin having approximately the equivalent bioactivity of the rapamycin.

In some embodiments, the rapamycin analogue is selected from the group consisting of biolimus (biolimus A9), 40-O-(2-Hydroxyethyl)-rapamycin (everolimus, RAD001), 40-O-Benzyl-rapamycin, 40-O-(4′-Hydroxymethyl)benzyl-rapamycin, [4′-(1,2-Dihydroxyethyl)]benzyl-rapamycin, 40-O-Allyl-rapamycin, 40-O-[3′-(2,2-Dimethyl-1,3-dioxolan-4 (S)-yl)-prop-2′-en-1 ‘-yl]-rapamycin, (2’: E,4'S)-40-O-(4′,5′-Dihydroxypent-2′-en-1′-yl)-rapamycin, 40-O-(2-Hydroxy) ethoxycar-bonylmethyl-rapamycin, 40-O-(3-Hydroxy) propyl-rapamycin, 40-O-(6-Hydroxy) hexyl-rapamycin, 0-[2-(2-Hydroxy) ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-4-[2-(N-Morpholino) acetoxy]ethyl-rapamycin, 40-O-(2-N-Imidazolylacetoxy)ethyl-rapamycin, 40-O-[2-(N-Methyl-N′-piperazinyl) acetoxy]ethyl-rapamycin, 39-O-Desmethyl-39,40-0,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, (2-Nicotinamidoethyp-rapamycin, 40-O-(2-(N-Methyl-imidazo-2′-ylcarbethoxamido)ethyl)-rapamycin, 40-O-(2-Ethoxycarbonylaminoethyl)-rapamycin, O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl) rapamycin (tacrolimus), 42-[3-hydroxy-(hydroxymethyl)-2-methylpropanoate]rapamycin (temsirolimus, CCI-779), ridaforolimus (AP-23573, MK-8669, deforolimus), (42S)-42-Deoxy-42-(1H-tetrazol-1-yl)-rapamycin (zotarolimus), and salts, derivatives, isomers, racemates, diastereoisomers, prodrugs, hydrate, ester, or analogs thereof.

In some embodiments, the rapamycin analogue is selected from the group consisting of temsirolimus (CCI-779), everolimus (RAD001), and ridaforolimus (AP-23573, MK-8669, deforolimus). In some embodiments, the rapamycin analogue comprises everolimus. In some embodiments, the rapamycin analogue comprises temsirolimus. In some embodiments, the rapamycin analogue comprises ridaforolimus.

As used herein, the term “administering” refers to bringing into contact with a compound of the present disclosure, such as rapamycin or rapamycin analogue. In some embodiments, the rapamycin or rapamycin analogue is administered locally. In some embodiments, the rapamycin or rapamycin analogue is administered systemically. Administration can be to animals, or organs or tissues, for example to the teat.

As used herein, the terms “administering”, “administer”, or “administration” refer to deliver the rapamycin or rapamycin analogue to an animal parenterally, enterally, or topically. Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intramammary infusion, intraspinal and intrasternal injection and infusion.

In some embodiments, the mTOR inhibitor is administered by intramammary infusion. In some embodiments, the rapamycin or rapamycin analogue is administered by intramammary infusion. In some embodiments, the rapamycin or rapamycin analogue is administered into the nipple. In some embodiments, the rapamycin or rapamycin analogue is administered into the teat. In some embodiments, the rapamycin or rapamycin analogue is administered into the teat canal. An artisan would appreciate that “intramammary infusion” may encompass infusion into the mammary gland, such that is used for treating intramammary infection (mastitis). An artisan would also be familiar with the techniques of performing such infusion, i.e. suitable syringe, cleaning and disinfecting the teat, milking out the udder prior to infusion, dispersing the product by gentle massage of the teat etc.

In some embodiments, the rapamycin or rapamycin analogue is administered in a composition, including a pharmaceutical composition. In some embodiments, the phrase “pharmaceutical composition” is employed herein to refer to those compounds, materials, compositions, combinations, and/or dosage forms which are, within the scope of sound medical judgment, suitable for use in contact with the tissues of animals without excessive toxicity, irritation, allergic response, or other problems or complications, commensurate with a reasonable benefit/risk ratio. In some embodiments, the composition may further comprise one or more vehicles, adjuvants and carriers.

In some embodiments, the composition may be in the form of a liquid, for example, an elixir, syrup, solution, emulsion or suspension. The liquid may be for delivery by injection. The liquid compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The composition can be enclosed in ampoules, disposable syringes or multiple dose vials made of glass or plastic. An injectable composition is preferably sterile.

An artisan would appreciate that a “milk-secretion stimulating amount” or a “milk-protein stimulating amount” may encompass, in some embodiments, a dosage sufficient to stimulate milk secretion or milk protein levels in the animal to which it is administered. The stimulation of milk secretion or milk protein levels may comprise, without limitation, the increase in production of milk extracted from an animal (amount or average amount) or the increase in the level of milk proteins over a period of time, compared to the milk production of animals which did not receive a milk-secretion stimulating amount of a mTOR inhibitor, such as rapamycin or a rapamycin analogue.

The dosage regimen for stimulating milk secretion is selected, in some embodiments, in accordance with a variety of factors, such as the type, age, weight, sex and medical condition of the animal, and the particular formulation employed, and thus may vary widely while still within the scope of the present disclosure.

Dosages may be titrated to optimize safety and efficacy. Typically, dosage-effect relationships from studies can provide useful guidance on the proper doses for animal administration.

In some embodiments, the rapamycin or rapamycin analogue is administered with one or more lipophilic vehicles. In some embodiments, the rapamycin or rapamycin analogue is administered at a dose of between 1 mg to 15 mg. In some embodiments, the rapamycin or rapamycin analogue is administered at a dose of between 2 mg to 20 mg. In some embodiments, the rapamycin or rapamycin analogue is administered at a dose of between 1 mg to 30 mg. In some embodiments, the rapamycin or rapamycin analogue is administered at a dose of between 16 mg to 30 mg. In some embodiments, the rapamycin or rapamycin analogue is administered at a dose of between 3 mg to 30 mg. In some embodiments, the rapamycin or rapamycin analogue is administered at a dose of between 2 mg to 10 mg. In some embodiments, the rapamycin or rapamycin analogue is administered at a dose of between 3 mg to 9 mg. In some embodiments, the rapamycin or rapamycin analogue is administered at a dose of at least 2 mg. In some embodiments, the rapamycin or rapamycin analogue is administered at a dose of at least 3 mg. In some embodiments, the rapamycin or rapamycin analogue is administered at a dose of at least 4 mg. In some embodiments, the rapamycin or rapamycin analogue is administered at a dose of up to 20 mg. In some embodiments, the rapamycin or rapamycin analogue is administered at a dose of up to 30 mg.

In some embodiments, the mTOR inhibitor is administered at a dose of about 1 mg, 2 mg, 3 mg, 4 mg, 5 mg, 6 mg, 7 mg, 8 mg, 9 mg, 10 mg, 11 mg, or 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, or 30 mg.

In some embodiments, the rapamycin or rapamycin analogue is administered at a dose of about 1 mg, 2 mg, 3 mg, 4 mg, 5 mg, 6 mg, 7 mg, 8 mg, 9 mg, 10 mg, 11 mg, or 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, or 30 mg. In some embodiments, the rapamycin or rapamycin analogue is administered at a dose of 1 mg. some embodiments, the rapamycin or rapamycin analogue is administered at a dose of about 2 mg. In some embodiments, the rapamycin or rapamycin analogue is administered at a dose of about 3 mg. In some embodiments, the rapamycin or rapamycin analogue is administered at a dose of about 4 mg. In some embodiments, the rapamycin or rapamycin analogue is administered at a dose of about 5 mg. In some embodiments, the rapamycin or rapamycin analogue is administered at a dose of about 6 mg. In some embodiments, the rapamycin or rapamycin analogue is administered at a dose of about 7 mg. In some embodiments, the rapamycin or rapamycin analogue is administered at a dose of about 8 mg. In some embodiments, the rapamycin or rapamycin analogue is administered at a dose of about 9 mg. In some embodiments, the rapamycin or rapamycin analogue is administered at a dose of about 10 mg. In some embodiments, the rapamycin or rapamycin analogue is administered at a dose of about 11 mg. In some embodiments, the rapamycin or rapamycin analogue is administered at a dose of about 12 mg. In some embodiments, the rapamycin or rapamycin analogue is administered at a dose of about 13 mg. In some embodiments, the rapamycin or rapamycin analogue is administered at a dose of about 14 mg. In some embodiments, the rapamycin or rapamycin analogue is administered at a dose of about 15 mg. In some embodiments, the rapamycin or rapamycin analogue is administered at a dose of about 16 mg. In some embodiments, the rapamycin or rapamycin analogue is administered at a dose of about 17 mg. In some embodiments, the rapamycin or rapamycin analogue is administered at a dose of about 18 mg. In some embodiments, the rapamycin or rapamycin analogue is administered at a dose of about 19 mg. In some embodiments, the rapamycin or rapamycin analogue is administered at a dose of about 20 mg. In some embodiments, the rapamycin or rapamycin analogue is administered at a dose of about 21 mg. In some embodiments, the rapamycin or rapamycin analogue is administered at a dose of about 22 mg. In some embodiments, the rapamycin or rapamycin analogue is administered at a dose of about 23 mg. In some embodiments, the rapamycin or rapamycin analogue is administered at a dose of about 24 mg. In some embodiments, the rapamycin or rapamycin analogue is administered at a dose of about 25 mg. In some embodiments, the rapamycin or rapamycin analogue is administered at a dose of about 26 mg. In some embodiments, the rapamycin or rapamycin analogue is administered at a dose of about 27 mg. In some embodiments, the rapamycin or rapamycin analogue is administered at a dose of about 28 mg. In some embodiments, the rapamycin or rapamycin analogue is administered at a dose of about 29 mg. In some embodiments, the rapamycin or rapamycin analogue is administered at a dose of about 30 mg. In some embodiments, the rapamycin or rapamycin analogue is administered at a dose of about 4.16 mg. In some embodiments, the rapamycin or rapamycin analogue is administered at a dose of about 8.32 mg.

In some embodiments, the mTOR inhibitor is administered daily. In some embodiments, the mTOR inhibitor is administered every other day (i.e. on alternate days, known also as skip-a-day dosing). In some embodiments, the mTOR inhibitor is administered once every 3 days. In some embodiments, the mTOR inhibitor is administered once every 4 days. In some embodiments, the mTOR inhibitor is administered once every 5 days. In some embodiments, the rapamycin or rapamycin analogue is administered daily. In some embodiments, the rapamycin or rapamycin analogue is administered every other day (i.e. on alternate days, known also as skip-a-day dosing). In some embodiments, the rapamycin or rapamycin analogue is administered once every 3 days. In some embodiments, the rapamycin or rapamycin analogue is administered once every 4 days. In some embodiments, the rapamycin or rapamycin analogue is administered once every 5 days.

In some embodiments, rapamycin is administered in 1, 2, 3, 4, 5, 6, 7, 8, 9, 10, 11, 12 or more times.

In some embodiments, the animal is further administered estrogen and progesterone.

In some embodiments, the animal is further administered estrogen. In some embodiments, the animal is further administered progesterone. In some embodiments, the animal is further administered estrogen and progesterone after rapamycin administration.

In some embodiments, the animal is further administered estrogen and progesterone after the rapamycin administration. In some embodiments, the animal is further administered estrogen and progesterone 1 day, 2 days, 3 days, 4 days, 5 days, 6 days, 7 days or more after rapamycin administration. In some embodiments, the animal is further administered estrogen and progesterone 4 days after the rapamycin administration.

In some embodiments, the animal is administered estrogen at a dose of between 0.01 mg to 1.0 mg/kg per day. In some embodiments, the animal is administered estrogen at 0.1 mg/kg per day. In some embodiments, the animal is administered progesterone at a dose of between 0.01 mg to 1.0 mg/kg per day. In some embodiments, the animal is administered progesterone at 0.25 mg/kg per day. In some embodiments, the animal is administered estrogen by subcutaneous injection. In some embodiments, the animal is administered progesterone by subcutaneous injection.

In some embodiments, the animal is further administered estrogen and progesterone after the rapamycin administration daily for 2 consecutive days. In some embodiments, the animal is further administered estrogen and progesterone after the rapamycin administration daily for 3 consecutive days. In some embodiments, the animal is further administered estrogen and progesterone after the rapamycin administration daily for 4 consecutive days. In some embodiments, the animal is further administered estrogen and progesterone after the rapamycin administration daily for 5 consecutive days. In some embodiments, the animal is further administered estrogen and progesterone after the rapamycin administration daily for at least 6 consecutive days. In some embodiments, the animal is further administered estrogen and progesterone after the rapamycin administration for at least 7 consecutive days.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

In the present disclosure the singular forms “a,” “an,” and “the” include the plural reference, and reference to a particular numerical value includes at least that particular value, unless the context clearly indicates otherwise. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable. In some embodiments, the term “about”, refers to a deviance of between 0.0001-5% from the indicated number or range of numbers. In some embodiments, the term “about”, refers to a deviance of between 1-10% from the indicated number or range of numbers. In some embodiments, the term “about”, refers to a deviance of up to 25% from the indicated number or range of numbers. The term “comprises” means encompasses all the elements listed, but may also include additional, unnamed elements, and it may be used interchangeably with the terms “encompasses”, “includes”, or “contains” having all the same qualities and meanings. The term “consisting of” means being composed of the recited elements or steps, and it may be used interchangeably with the terms “composed of” having all the same qualities and meanings.

It should be understood that the disclosure presented herein is not limited to the particular methodologies, protocols and reagents, and examples described herein. The terminology and examples used herein is for the purpose of describing particular embodiments only, for the intent and purpose of providing guidance to the skilled artisan, and is not intended to limit the scope of the disclosure presented herein.

### EXAMPLES

### Example 1

Goal: analyze rapamycin's effect on mammary epithelial stem cell self-renewal and the potential consequences on development and milk production.

### Methods:

### Animals

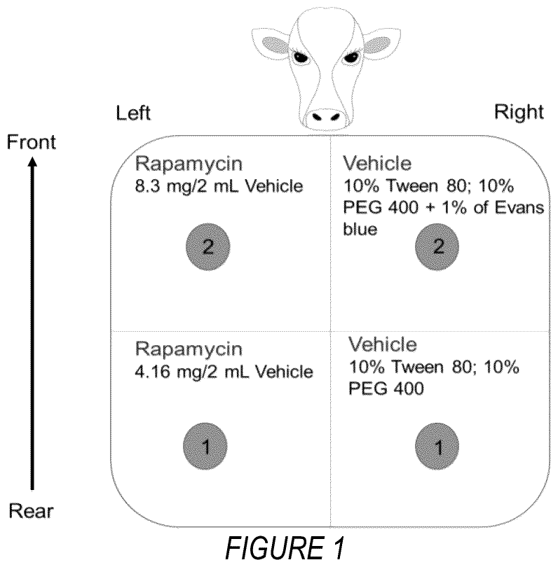

Three-month-old female Holstein calves (˜90 kg body weight [BW]) that were raised on the Volcani Center's experimental farm were used in these experiments. During the experimental periods, each mammary gland was administered via the teat canal with 2 mL of vehicle, composed of sterile PBS containing 10% polyethylene glycol (Sigma, St. Louis, MO) and 10% Tween 80 (Sigma). In individual experiments, the vehicle was supplemented with Evans blue to analyze the penetration of this solution into the mammary epithelium, or with rapamycin to analyze its effect on cellular activities. The vehicle was administered using a sterile 22G IV cannula (Deltaven, Viadana, (MN) Italy), cut to ˜1.5 cm and inserted (without the needle) into the teat canal. This procedure did not involve any sedation or anesthesia. In the relevant experiments, estrogen (0.1 mg/kg per day, Sigma) and progesterone (0.25 mg/kg per day, Sigma) were administered following the rapamycin treatment by daily subcutaneous injections for 4 consecutive days. The design of the studies was based on the distinct morphology and activity of the individual “quarters” (i.e., glands) of the bovine udder which can be individually affected. Thus, in studies analyzing the rapamycin effects, front or rear glands of each side of the calf served as internal controls for the other two rapamycin-administered glands. The dye Evans blue was added to one of the vehicle-treated glands to follow its penetration into the epithelial morphology in close and far regions from the teat (FIG. 1).

At the end of the experimental period, calves were heavily sedated with intravenous xylazine (20 mg/mL; 0.1 mg/kg, Sedaxylan Veterinary, Abic Phibro, Bet Shemesh, Israel) and ketamine (1 g/10 mL; 0.5 mg/kg, Clorketam, Vetoquinol, Lure, France) and sacrificed with intravenous 20% embutramide, 5% mebezonium iodide and 0.5% tetracaine hydrochloride (T-61; 0.15 mL/kg, Intervet Canada Corp., Quebec, Canada). The complete udder was then removed for further analyses.

### Morphological Analysis by Whole-Mount Examination

For whole-mount examination, bovine mammary parenchymal tissue pieces (˜0.5 cm3) were excised from the udder, fixed, and stained with Carmine red. Stained whole mounts were visualized and photographed using a binocular equipped with cellSens standard 1.4 software (Olympus Scientific Solutions, Tokyo, Japan).

### Histological Analysis, Immunohistochemistry and Immunofluorescence

Bovine mammary samples were fixed in Bouin's solution, dehydrated in a graded ethanol series (50% to 100%), cleared in xylene and embedded in paraffin. Paraffin sections (5 μm) were stained with hematoxylin and eosin (H&E; Sigma) to visualize the morphology of the mammary layers.

Immunohistochemistry was performed on the 5-μm paraffin-embedded sections after antigen retrieval. The reaction with primary antibodies against β-casein (Barash lab), pS6 (Cell signaling Inc.), CK18 (GeneTex), and PCNA (BioLegend), was initiated by blocking the sections with 10% bovine serum albumen and 5% goat serum in 0.1% PBST for 1 h prior to overnight incubation with the first antibody at 4° C. Sections were washed and incubated with N-Histofine (Nichirei Biosciences, Tokyo, Japan) for 30 min at room temperature. Signals were generated with 3,3-diaminobenzidine (DAB) substrate (Vector Laboratories, Burlingame, CA).

For immunofluorescence analyses using antibodies against aSMA (Santa Cruz) and CK18 (GeneTex), the sections were incubated with secondary antibodies for 1 h at room temperature. Nuclei were stained with 4′,6-diamidino-2-phenylindole (DAPI) (Qbiogen, Irvine, CA). In all of the analyses, stained tissues were visualized and photographed under an inverted fluorescence microscope (Eclipse Ti, Nikon Instruments, Melville, NY) equipped with NIS-Elements AR 3.2 imaging software (Nikon Instruments), or an Olympus IX 81 inverted laser scanning confocal microscope (FluoView 500, Tokyo, Japan).

### Flow Cytometry

Bovine mammary tissue pieces were harvested from the parenchymal region of each gland, digested into organoids and kept at −80°. For flow cytometry, organoids were washed and further digested into dispersed mammary cells. Lin-(CD45, TER119, CD31 and BP-1) cell suspension was prepared using the EasySep mouse epithelial enrichment kit according to the manufacturer's protocol (STEMCELL Technologies, Vancouver, Canada), labelled with PE-conjugated anti-CD24 and FITC-conjugated anti-CD49f antibodies. Cell viability was tested by staining with BD Horizon Fixable Viability Stain 450 diluted 1:500 (BD Biosciences, San Jose, CA). Cell sorting was performed in a FACSAria III cell sorter (BD Biosciences) at the Department of Biological Services of the Weizmann Institute of Science (Rehovot, Israel). RNA extraction and real-time PCR analyses

RNA was extracted from the parenchymal region of each mammary gland using TRIzol reagent and reverse-transcribed with the qScript Synthesis Kit (Quanta BioSciences, Beverly, MA). RNA quality and quantity were determined in a NanoDrop 1000 spectrophotometer (Thermo Fisher Scientific, Wilmington, DE). Quantitative real-time PCR analyses were performed in a StepOnePlus instrument (Applied Biosystems, Foster City, CA) in a 10-mL reaction volume containing 1 μL cDNA, 5 μL PerfeCta SYBR Green FastMix, Rox (2X) (Quanta BioSciences) and 0.5 μL of the primers listed in Table 1. The thermal cycling conditions consisted of 20 s at 95° C. followed by 40 cycles of 3 s at 95° C. and 30 s at 60° C. The amplification curves for the selected genes were parallel. The gene coding for elF4E or ribosomal S16 was used as the endogenous control. Fold change (relative expression) was calculated using StepOne v2.1 and DataAssist v2.0 software (Applied Biosystems).

TABLE 1

| | | | SEQ |
|---|---|---|---|
| | | | ID |
| Primer | Gene ID | Sequence | NO. |
| α-Casein S1 | 282208 | AAGAGGGAATCCATGCCCAA | 1 |
| Forward | | | |
| α-Casein S1 | 282208 | AGGCCAGTTCCTGATTCACTC | 2 |
| Reverse | | | |
| DELTA 1 (DLL1) | 788775 | AGGGCCAGTACTGCACAGA | 3 |
| Forward | | | |
| DELTA 1 (DLL1) | 788775 | AAAATCCGTGCTGCTCATC | 4 |
| Reverse | | | |
| ERα (ESR1) | 407238 | CGCAAGTGCTATGAAGTGG | 5 |
| Forward | | | |
| ERα (ESR1) | 407238 | CGCTTGTGCTTCAACATTC | 6 |
| Reverse | | | |
| STAT5A Forward | 282375 | GCAGCCATCTCGAGGACTA | 7 |
| STAT5A Reverse | 282375 | GAACCACTGCCAGAAGGTG | 8 |
| α-lactalbumin | 281894 | CTCCAGGGGTGCATGAATGG | 9 |
| Forward | | | |
| α-lactalbumin | 281894 | TAAAAGCGCCATCAGGGACAT | 10 |
| Reverse | | | |
| BLG Forward | 100848610 | GGGAAAACCACGAGTGTGTG | 11 |
| BLG Reverse | 100848610 | TTTCCCCGTGATAGTTGACCG | 12 |
| EIF4E Forward | 281751 | CAGGAGGTTGCTAACCCAGA | 13 |
| EIF4E Reverse | 281751 | TGCTTGCCAAGTTTTGCTT | 14 |
| Ki67 Forward | 513220 | GTCAGCAGCTTCGGTGATT | 15 |
| Ki67 Reverse | 513220 | GAGACCCGCCTCCTCTTT | 16 |
| Notch1 Forward | 767866 | AACGAGTTCGTGTGCGAGT | 17 |
| Notch1 Reverse | 767866 | GTTCTTGCAGGGTGTGCTT | 18 |
| Jagged 1 | 783681 | TCCCACTGGTTTCTCTGGA | 19 |
| Forward | | | |
| Jagged 1 | 783681 | CTGGCTCGGTTGTAGCACT | 20 |
| Reverse | | | |
| LGR4 Forward | 505423 | GGGCTTAAAAGCACCTGTCGA | 21 |
| | | A | |
| LGR4 Reverse | 505423 | AGGTCTCAGTGATGTGGCAGG | 22 |
| LGR5 Forward | 520189 | AGTTGTTCAGCCTCCGATCT | 23 |
| LGR5 Reverse | 520189 | TGGAAAATGCATTAGGGTCA | 24 |
| S16 Forward | 506297 | ATCTCGTCTCTGCGCCTTGA | 25 |
| S16 Reverse | 506297 | GCATTCTTCTGCCGGTGTCG | 26 |
| LGR6 Forward | 100336662 | GTGTCCTGGAGCTGTCTCAC | 27 |
| LGR6 Reverse | 100336662 | CTCCTCCAGCTTCTGACACC | 28 |
| β-casein | 281099 | TTCCATTGCCTGGACTACTTG | 29 |
| Forward | | T | |
| β-casein | 281099 | CAACAGCCAAAAGGCAGGTA | 30 |
| Reverse | | | |

### Immunoblot Analysis

Proteins from the parenchymal region of each mammary gland were extracted with RIPA buffer containing 10 mM Tris-HCl pH 7.5, 150 mM NaCl, 0.1% SDS, 1% sodium deoxycholate and 5 mM EDTA (Sigma), protease inhibitor cocktail and phosphatase inhibitor cocktail II (both from Sigma). Equal amounts of protein, determined by QPRO-BCA Kit Standard (Cyanagen, Bologna, Italy) according to the manufacturer's protocol, were subjected to SDS-PAGE. Fractionated proteins were transferred to nitrocellulose filters and the uploading of equal amounts of protein per sample was confirmed by staining the blot with Ponceau S (Sigma). The expression levels of phosphorylated S6 (pS6) and β-actin were determined according to the signals generated with their respective antibodies. Signals were generated with WESTAR Supernova (Cyanagen, Bologna, Italy) combined with Super Signal chemiluminescent substrate (Thermo Fisher Scientific), and detected in a Syngene analyzer (Cambridge, UK).

### Analysis of Propagation Rate

For propagation rate, cells from each treatment were seeded in 96-well cell-culture plates (Corning, Corning, NY) at a density of 10,000 cells/well and cultured in mammary medium composed of DMEM-F12 medium containing 5% FBS, hydrocortisone (0.5 mg/ml, Sigma), insulin (5 mg/ml, Sigma), gentamicin (50 mg/ml, Biological Industries), streptomycin and penicillin (100 mg/ml and 100 U/ml, respectively), hEGF (10 ng/ml, Merck, Darmstadt, Germany), hFGF2 (10 ng/ml, Merck), heparin (4 mg/ml, Merck), cholera toxin (10 ng/ml, Sigma) and B27 (4 ml stock/ml, Invitrogen, Carlsbad, CA) (10) for the indicated period.

### Organoid Cultures

Partially digested pieces of parenchymal tissue were cultured on inserts (40 μm Falcon nylon cell strainers, Corning) in 6-well plates and supplemented with mammary medium. Rapamycin (10 ng/mL) was administered to selected wells every other day. After a 3-week period, rapamycin-treated organoids were washed, and all cultures were administered mammary medium for a week to avoid any further direct rapamycin effect. Medium was replaced every day. Eventually, the serum- and growth factor-containing medium was washed, and milk protein gene expression was induced during 5 consecutive days of culture in DMEM/F12 medium containing (only) insulin (5 μg/mL), hydrocortisone (1 μg/mL) and prolactin (3 μg/mL). At the end of the culture period, organoids were frozen in liquid nitrogen and kept at −80° C.

### Statistics

Unless otherwise indicated, t-test was performed for statistical analyses. Correlations were calculated using the Excel calculation formula.

### Results:

### Establishment and Calibration of Rapamycin Intramammary Administration Methodology

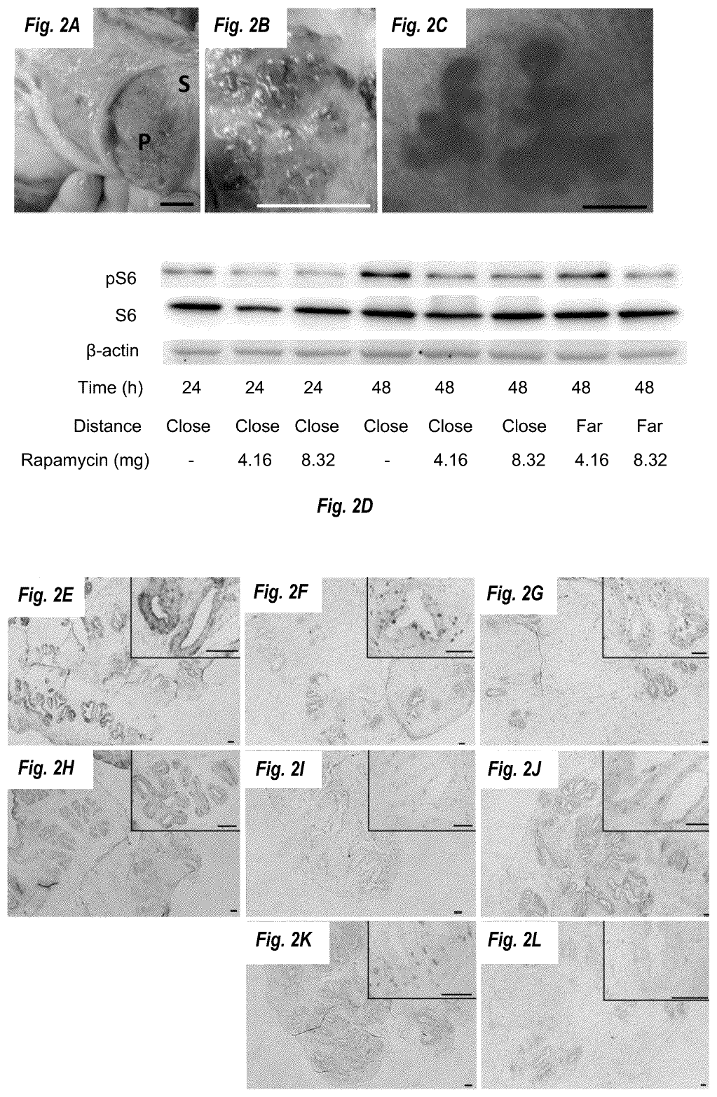

The intramammary drug-delivery method to the three-month-old female calves that engages the milk-mobilizing ductal system was preferred over systemic administration, to selectively confine rapamycin's effect to the mammary parenchyma (FIG. 2A). To monitor vehicle mobility and penetration into the mammary epithelium, Evans blue (1%) was dissolved in the lipophilic solution and administered via the nipple to each of the four glands. Three hours later, the calf was sacrificed, the udder was removed and blue-stained parenchyma was revealed under the skin (FIG. 2B). In the short time interval, about half of the injected volume (˜1 mL) was still located in the cisterna and did not penetrate the ductal system. Nevertheless, analysis of Carmine-red-counterstained mammary whole mounts revealed penetration of the blue stain into the ductal system and its translocation in the mammary epithelial terminal ductal lobuloalveolar units (TDLUs) the ductal elongation region (FIG. 2C).

To determine an effective concentration and administration interval, mammalian target of rapamycin (mTOR) responsiveness to rapamycin administration was studied in parenchymal regions that were close to and far from the teat. Rapamycin was delivered to the parenchymatic region of two calves at two concentrations (4.16 or 8.32 mg/gland per day). The calves were sacrificed 24 h or 48 h later and the udders were removed. Rapamycin administration did not seem to have any effect on tissue morphology in the various parenchymatic regions tested. Table 2 shows S6 phosphorylation in the mammary parenchyma from analysis of immunoblot (FIG. 2D) and immunohistochemistry (FIGS. 2E-2L). These data show that suppression of S6 phosphorylation following rapamycin administration at a level of 8.32 mg/2 mL in regions close to and far from the teat was maintained for at least 48 h. Note that complete repression of mTOR activity was avoided in these experiments to preserve the self-renewing capability of the epithelial stem cells.

TABLE 2

| | Time after infusion (h) | | | | | | | |
|---|---|---|---|---|---|---|---|---|
| | 24 | 24 | 24 | 48 | 48 | 48 | 48 | 48 |
| Distance | Close | Close | Close | Close | Close | Close | Far | Far |
| from nipple | | | | | | | | |
| Rapamycin | — | 4.16 | 8.32 | — | 4.16 | 8.32 | 4.16 | 8.32 |
| (mg) | | | | | | | | |
| pS6/S6 | 0.47 | 0.31 | 0.39 | 0.76 | 0.54 | 0.58 | 0.71 | 0.37 |
| | (100%) | (66%) | (95%) | (100%) | (71%) | (76%) | (93%) | (48%) |
| pS6/β-actin | 1.71 | 1.29 | 1.08 | 3.15 | 2.03 | 2.61 | 2.14 | 1.77 |
| | (100%) | (75%) | (63%) | (100%) | (64%) | (81%) | (68%) | (56%) |

### Rapamycin Administration Decreases Mammary S6 Phosphorylation and Induces Indicators of Stem Cell Self-Renewal

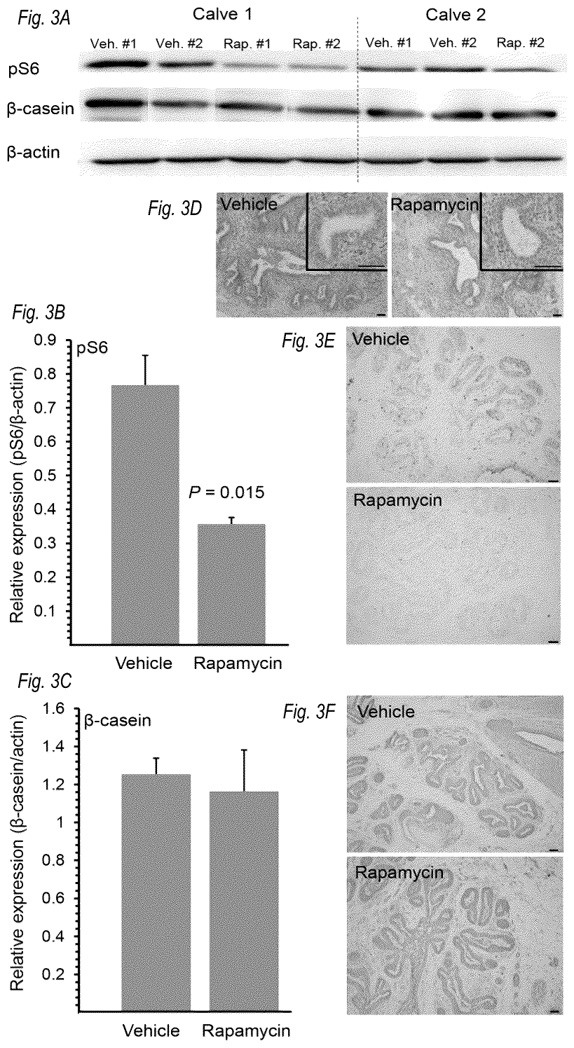

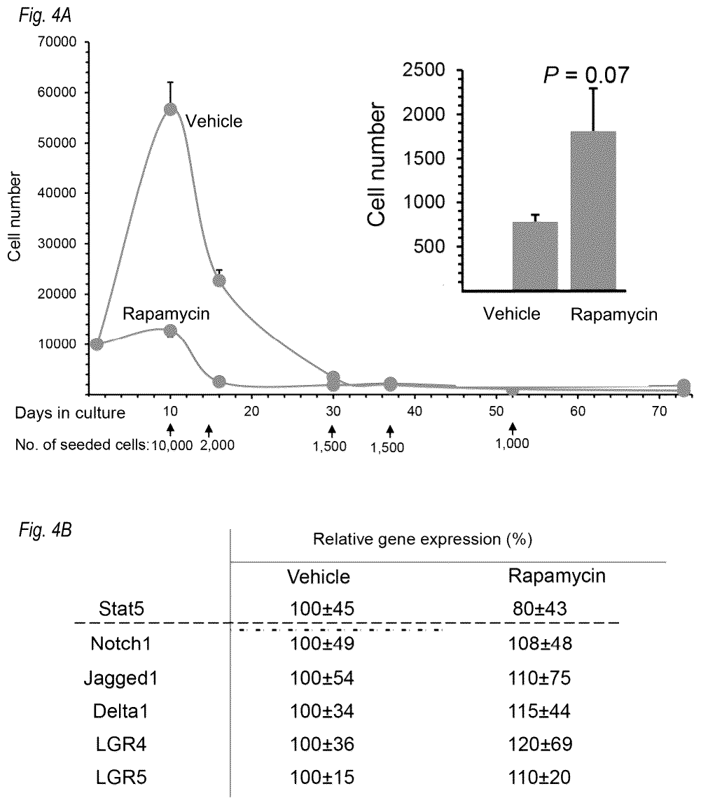

The effect of 3 weeks of skip-a-day rapamycin administration (8.32 mg/gland per day) on stem cell self-renewal (FIGS. 3A-3F) was examined. Here, a significant decrease of 53% in S6 phosphorylation was observed by immunoblot analysis, 48 h after termination of the experiment (FIGS. 3A and 3B), which was not associated with any detectable effect on β-casein synthesis (FIG. 3C) or mammary gland morphology (FIG. 3D). Immunohistochemical analyses localized the decreased pS6 expression exclusively to the mammary epithelial cells (FIG. 3E) and confirmed comparable β-casein synthesis in vehicle- and rapamycin-treated glands (FIG. 3F).

Rapamycin's effect on cell-propagation rate was analyzed in cultures from individual vehicle- and rapamycin-treated glands (FIG. 4A). This method produces a clear and significant distinction of the bovine mammary epithelial stem cell population from the more differentiated ones.

Here, a significant induction in cell number was initially detected in cultures from the vehicle-treated glands, but not in their rapamycin-treated counterparts. This probably represented propagation of partially differentiated epithelial cells with a relatively short survival rate, as evidenced by the consequent decrease in cell number. Indeed, by day 30 of culture, the number of cells from the vehicle-treated and rapamycin-treated glands equalized. In the long-term, higher propagation rate was observed for cultures from the rapamycin-treated gland, starting from 1.15-fold and 1.4-fold differences on days 37 and 52 of culture, respectively, and progressing to a 2.6-fold difference at the end of the culture period (FIG. 4A inset).

There is no individual marker that specifically identifies bovine mammary stem cells. Nevertheless, a collection of genes that are highly expressed in mouse and human mammary stem cells were highly expressed in the rapamycin-treated glands, by 8-20% more than in their vehicle-treated counterparts (FIG. 4B). In contrast, Stat5, which serves as a reliable reference for luminal progenitors, was expressed at lower levels in the rapamycin-treated glands. Due to the high variability in expression among mammary glands, none of these results were significant at P<0.05.

### Higher Cell Proliferation in Rapamycin-Treated Glands Depends on Proximity to the Growing Site and Studied Cell Layer

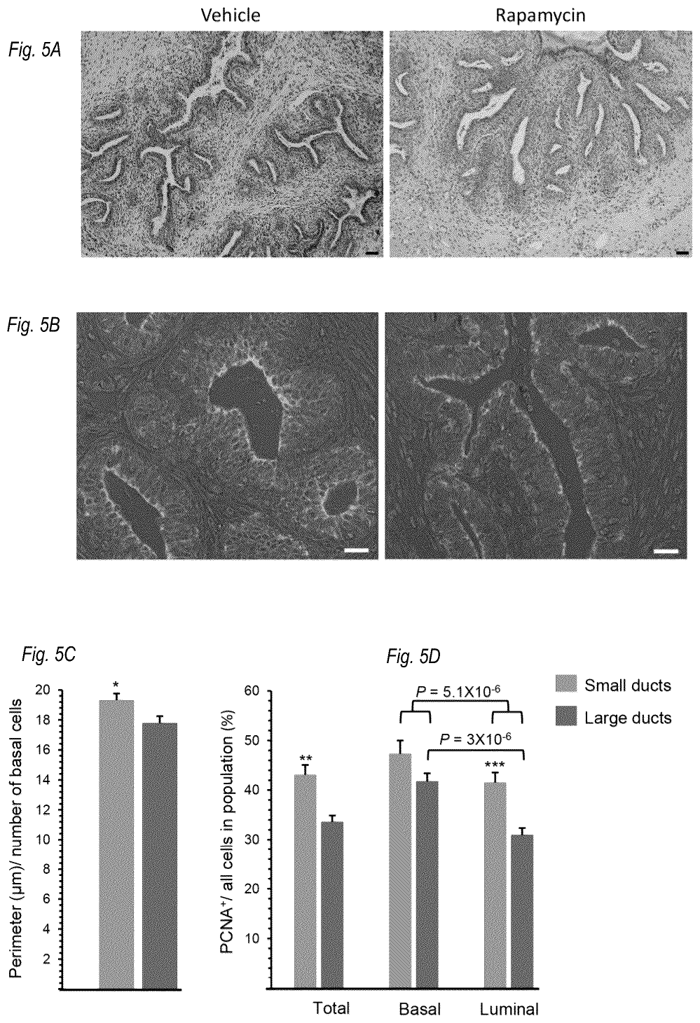

To study the effect of rapamycin administration and induced stem cell self-renewal on mammary cell proliferation and milk protein expression, front and rear mammary glands of three calves (six for each treatment) were infused with either vehicle or rapamycin for 3 weeks (8.3 mg/gland per day). Five days after the last rapamycin administration, calves were subjected to daily estrogen and progesterone administration for 4 consecutive days to reproduce mammary cell proliferation during pregnancy. At the end of the experimental period, animals were sacrificed, udders were removed and mammary parenchymal tissue pieces (˜0.5 cm3) were subjected to H&E staining and immunofluorescence analyses (FIG. 5A and FIG. 5B). Ducts of various diameters were identified that penetrated the stroma and encompassed a multilayered luminal epithelium stained with CK18, lined by a single layer of basal myoepithelial cells stained with alpha smooth muscle actin (aSMA) (FIG. 5B). There were no apparent differences in morphology between the vehicle- and rapamycin-treated glands. Tukey-Kramer (means comparison) statistical analysis ruled out any preferential effect on ductal cell number or diameter for an individual animal or gland. This enabled a more detailed characterization of the experimental system, by analyzing ducts in all vehicle-treated glands with a diameter of 51.35-191.81 μm (larger sizes did not enable distinguishing branching ducts from small independent ones; in smaller ducts, distinction of basal cells from stromal and immune cells was challenging). A cross section of an average-sized duct in a 3-month-old calf appeared to be comprised of 84 cells that interact to create a perimeter of ˜350 μm (Table 3). Luminal cells represented most of the epithelial cell population, whereas the single-layered basal/myoepithelial cells made up only ˜25% of the total cell number. Morphologically, these basal cells were significantly (P<0.05) more densely packed in the perimeter of the large ducts (diameter >100 μm) compared to the small ones (diameter <100 μm) (FIG. 5C).

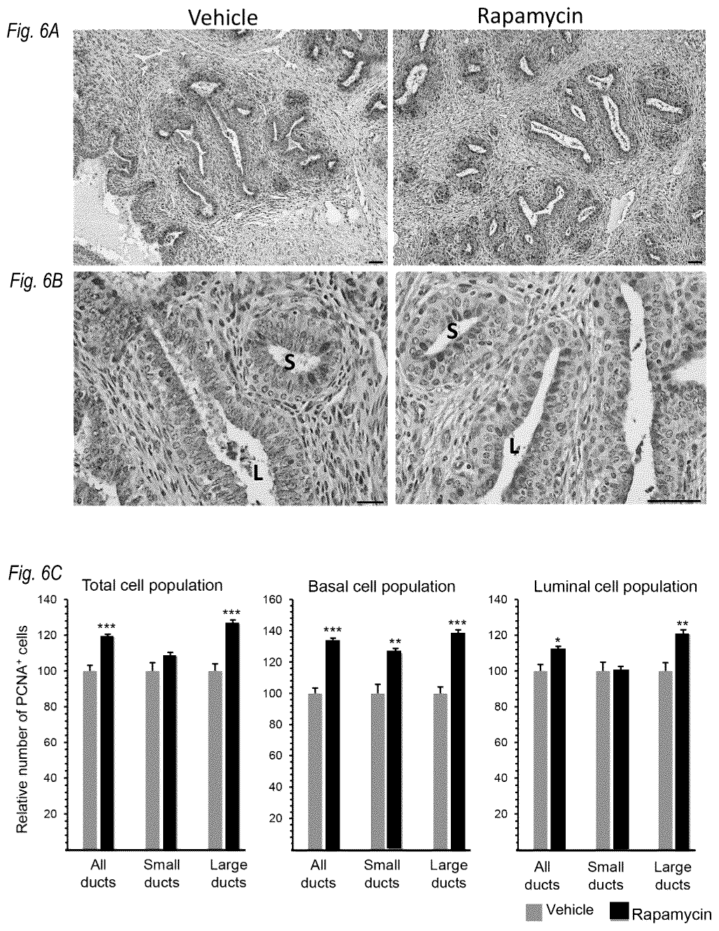

To monitor cell proliferation, parenchymal sections from the individual mammary glands were stained with proliferating cell nuclear antigen (PCNA; FIG. 6A and FIG. 6B). About 45% of the total number of cells in the vehicle-treated glands stained PCNA+ (Table 3). The relative rate of basal cell proliferation was 1.26-fold higher (P<5.1×10-6) than that of the luminal ones (FIG. 5D).

TABLE 3

| | Average ducts | | | Small ducts | | | Large ducts | | |
|---|---|---|---|---|---|---|---|---|---|
| | Diameter/perimeter | | | | | | | | |
| | 111.3 ± 2.3/349.7 ± 7.4 | | | 85.4 ± 1.87/268.21 ± 5.8 | | | 125.7 ± 2.3/394.7 ± 7.2 | | |
| | Total | Basal | Luminal | Total | Basal | Luminal | Total | Basal | Luminal |
| Number of | 84.3 ± 3.3 | 20.2 ± 0.7 | 63.9 ± 2.7 | 53.2 ± 2.0 | 14.3 ± 0.5 | 36.6 ± 1.6 | 101.2 ± 3.8 | 38.6 ± 1.6 | 77.5 ± 3.3 |
| cells | | | | | | | | | |
| % of total | 100 | 25.3 ± 0.5 | 74.6 ± 0.5 | 100 | 27.7 ± 0.7 | 72.2 ± 0.7 | 100 | 24.2 ± 0.6 | 75.7 ± 0.6 |
| PCNA + | 28.7 ± 1.1 | 8.5 ± 0.3 | 20.2 ± 0.2 | 21.7 ± 0.92 | 6.5 ± 0.3 | 15.2 ± 0.7 | 32.4 ± 1.4 | 9.6 ± 0.4 | 22.8 ± 1.2 |
| cells | | | | | | | | | |

Separate analyses were conducted for ducts with small and large diameters (FIG. 5D), representing regions close to and far from the TDLUs, respectively. General proliferation rate was 28% higher in the small vs. large ducts, mainly due to 31% higher proliferation of luminal cells. Interestingly, in small ducts, no significant (P<0.05) differences could be detected between the proliferation rates of basal and luminal cells, whereas in the large ducts, basal cell proliferation was 37% higher than that of luminal cells.

Rapamycin administration increased the total number of PCNA+ cells by 20%, mainly due to a significant effect on cells composing the large ducts (FIG. 6C). A more detailed analysis indicated that basal cell proliferation was induced by rapamycin in both small and large ducts, whereas a markedly higher rate of PCNA+luminal cells was detected only in the large ducts of the rapamycin-treated glands.

### Milk Protein Gene Expression is Induced in the Rapamycin-Treated Glands, and is Negatively Correlated to Endogenous Milk Protein Expression Potential

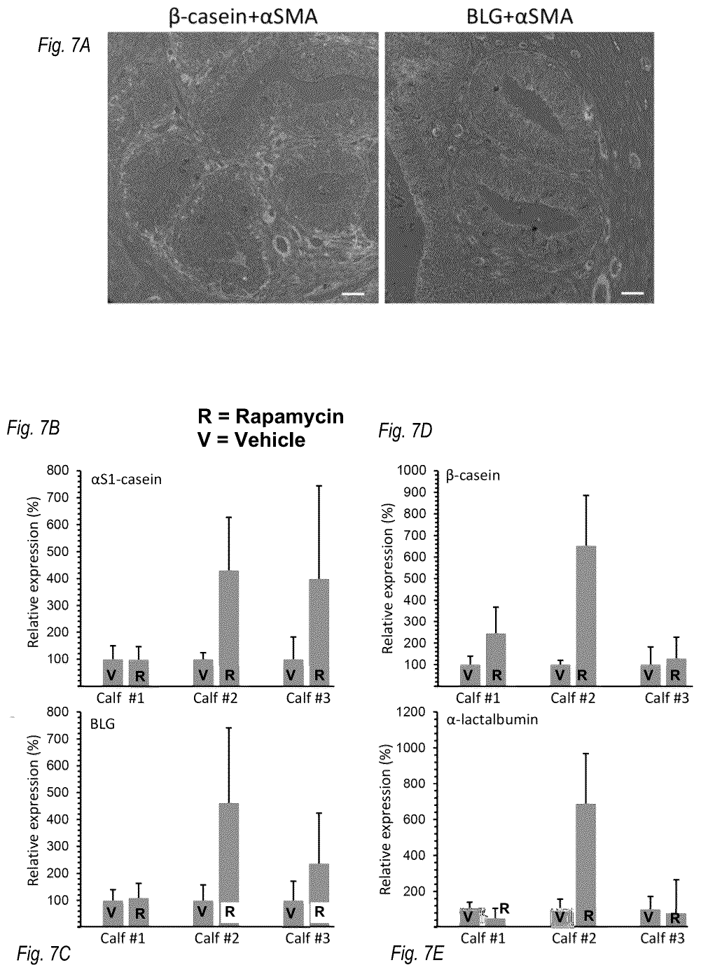

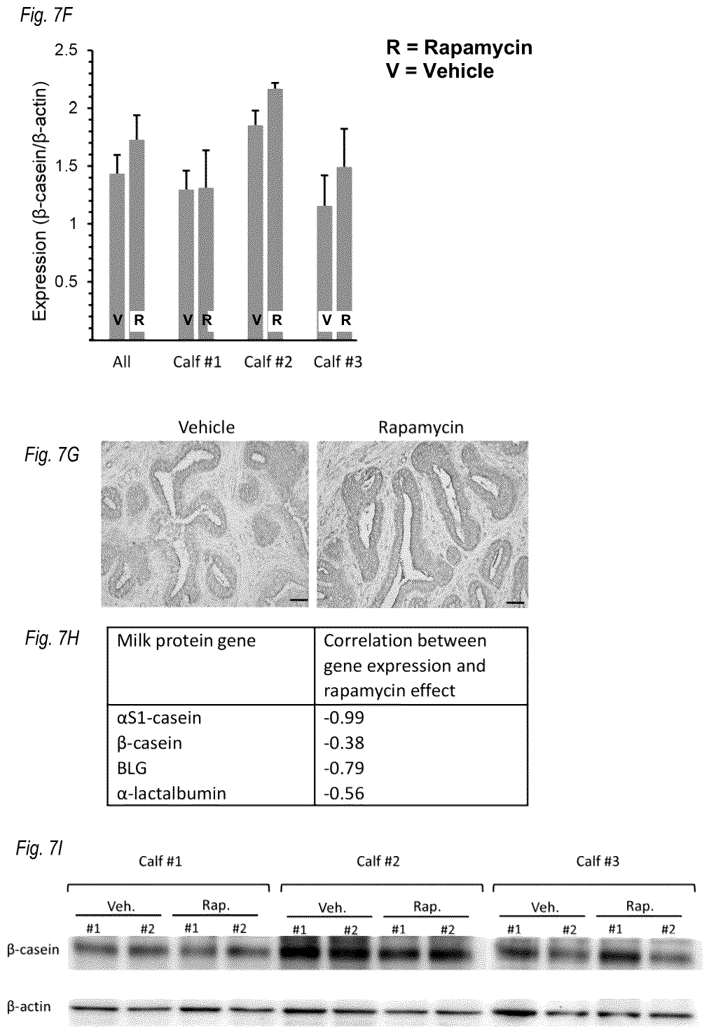

The latent effect of rapamycin administration on milk protein gene expression and synthesis was measured in the calves' mammary glands (FIGS. 7A-7I). The detectable expression of β-casein and β-lactoglobulin (BLG) in luminal mammary epithelial cells (FIG. 7A) warranted more accurate gene and protein analyses of mammary productivity. Thus, expression of aS1-casein, β-casein, BLG and α-lactalbumin was analyzed by comparing the relevant gene expression in the rapamycin-treated glands of each animal to that in the vehicle-treated ones. In calf #2, rapamycin administration resulted in a latent 4.5- to 6-fold induction in the expression of all four milk protein genes tested, with no difference between the responses of casein and whey protein genes (FIG. 7B-FIG. 7E). This positive effect was confirmed by immunohistochemical analysis of β-casein, localized in the luminal epithelial cells (FIG. 7G), and by immunoblot analysis (FIG. 7F and FIG. 7I). A modest effect of rapamycin on aS1-casein and BLG expression was observed in calf #3, but not in calf #1. Importantly, the various milk protein genes were expressed by mammary glands of calves #2 and #3 at levels of 0.4-12%, as compared to mammary glands of calf #1, which maintained much higher expression levels. Associating the average endogenous expression levels of the individual milk protein genes in the two vehicle-treated glands of each of the three calves to the inductive rapamycin effect (fold change), measured by comparing average gene expression in the two rapamycin-treated glands to their respective controls, presented a negative correlation of up to 99% for each gene (FIG. 7H). Taken together, these results suggest that the positive rapamycin effect is limited by the endogenous expression potential of the mammary gland.

Conclusions: Using Evans blue marker dissolved in a lipophilic vehicle and counterstaining with Carmine red, intraductal delivery of material to the epithelium was established as an alternative to systemic administration. Advantageously, targeting mammary epithelial cells around the ductal lumen, including those that are relatively far from the nipple, requires much less material compared to systemic administration and eliminates side effects resulting from the involvement of other organs.

Intramammary rapamycin administration was calibrated according to S6 phosphorylation in the epithelial cells. The amount and methodology chosen here for rapamycin administration did not have pathological consequences, but was able to induce stem cell self-renewal as depicted by the longer propagation rate of cultures originating from rapamycin-treated glands compared to their respective controls, and the collective increase in stemcell marker expression.

Four days of estrogen and progesterone administration are sufficient for initial induction of bovine mammary cell proliferation. Temporally constrained by ethical regulations, this steroid-administration procedure was reproduced here to follow the consequences of rapamycin administration during late pregnancy.

Rapamycin administration resulted in latent induction of cell proliferation. Rapamycin induced cell proliferation selectively, discriminating large ducts with basal and luminal cell responses from small ducts in which only luminal cell proliferation is induced. This selectivity may be partly associated with the higher density of basal myoepithelial cells in the large ducts (i.e., far from the TDLUs) which may tightly control their proliferation.

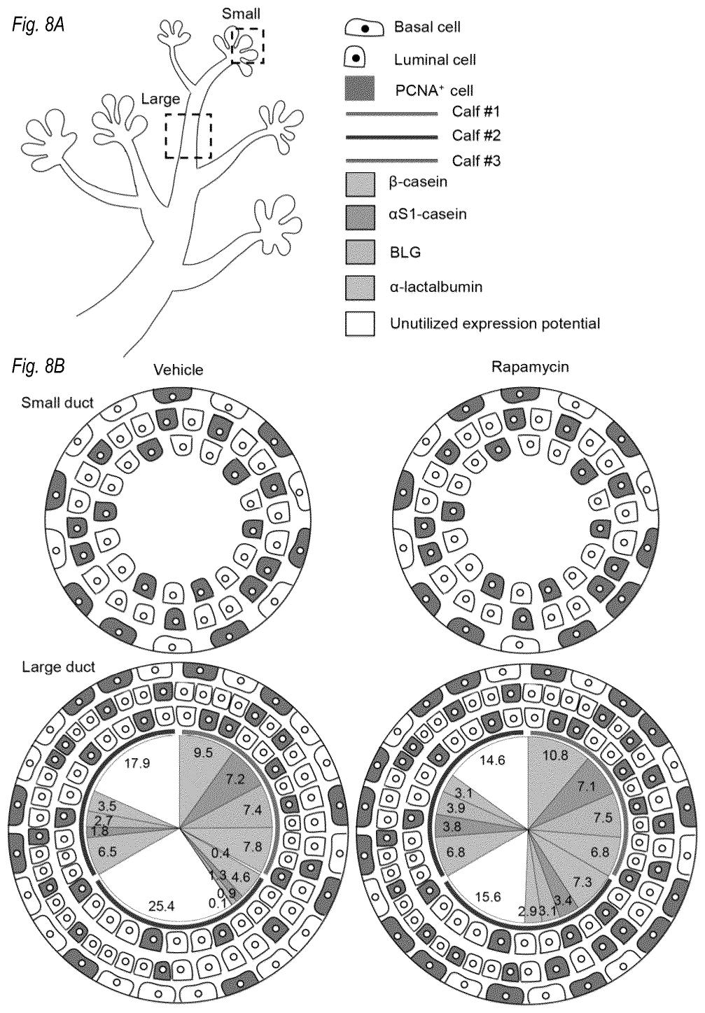

The bovine mammary parenchyma seems to contain sufficiently differentiated epithelial cells at the age of 3 months to allow milk protein gene expression and protein synthesis. This suggests that the full repertoire of systemic and niche-dependent stimuli necessary for full differentiation of the epithelial stem cell toward a functional luminal cell already exists at this early developmental stage. Their activity in prepuberty dictates future productivity of the mammary gland. Rapamycin's positive effect on milk protein gene expression varied among calves (FIGS. 8A-8B) was negatively related to the gland's endogenous expression potential. In addition, the number of proliferating epithelial cells in the mammary gland was also induced by rapamycin administration as determined by PCNA staining that detects only proliferating cells. The latent increase in the number of proliferating cells after rapamycin administration in the epithelial is depicted in FIGS. 8A-8B.

1. A method of increasing the milk production of an animal or increasing the level of milk proteins in an animal, the method comprising administering to the animal a milk-secretion stimulating amount of a mTOR inhibitor.

2. (canceled)

3. The method of claim 1, wherein the mTOR inhibitor comprises rapamycin or a rapamycin analogue.

4. The method of claim 1, wherein the mTOR inhibitor is administered by intramammary infusion.

5. The method of claim 1, wherein, the mTOR inhibitor is administered at a dose of between 1 mg to 30 mg.

6. The method of claim 1, wherein the animal is further administered estrogen and progesterone.

7. The method of claim 1, wherein said milk protein is selected from the group consisting of α-S1-casein (alpha-S1-casein), α-S2-casein (alpha-S2-casein), β-casein (beta-casein), κ-casein (kappa-casein), β-lactoglobulin (BLG), and α-lactalbumin (alpha-lactalbumin).

8. The method of claim 1, wherein the animal is a mammal.

9. The method of claim 1, wherein the animal is a non-lactating animal.

10. The method of claim 1, wherein the animal is a dairy heifer or a non-pregnant cow.

11. The method of claim 1, wherein the animal is a dairy livestock animal.

12. The method of claim 11, wherein the livestock animal is selected from the group consisting of a heifer, a cow, a goat, a sheep and a water buffalo.

13. The method of claim 1, wherein the animal is a calf.

14. The method of claim 1, wherein the animal is a 3-month-old calf.

15. A method for inducing epithelial cell proliferation in the mammary gland of an animal comprising administering to the animal a cell proliferation stimulating amount of a mTOR inhibitor.

16. The method of claim 15, wherein said epithelial cells comprise luminal epithelial cells.

17. The method of claim 15, wherein said epithelial cells comprise myoepithelial (basal) cells.
